(19) United States
(12) Patent Application Publication
(10) Pub. No.: US 2010/0239665 A1
(43) Pub. Date: Sep. 23, 2010

(54) PHARMACEUTICAL NIMODIPINE COMPOSITIONS

(76) Inventor: Ivan Coulter, Dublin (IE)

Correspondence Address: KLARQUIST SPARKMAN, LLP, 121 SW SALMON STREET, SUITE 1600, PORTLAND, OR, 97204, US

(21) Appl. No.: 12/598,395

(22) PCT Filed: May 1, 2008
(86) PCT No.: PCT/IE08/00051

§ 371 Date: Apr. 30, 2010

Related U.S. Application Data
(60) Provisional application No. 60/924,132, filed on May 1, 2007.

(51) Int. Cl. A61K 9/52; A61K 31/44; A61K 9/14

(52) U.S. Cl. 424/457; 514/356; 424/489

(57) ABSTRACT

A modified release solid dosage product comprises a plurality of minicapsules or minispheres containing nimodipine, wherein when exposed to a use environment more than 40% of the nimodipine is released within 12 hours and wherein the Tₘₐₓ is reached within 6 hours. The product may be a capsule 4 having a first population of uncoated minispheres 1 containing nimodipine for immediate release and a second population of coated minispheres 2 containing nimodipine for sustained release. There may be another population of coated minicapsules 3.

Nimodipine, a member of the dihydropyrimidine class of drugs, belongs to the class of pharmacological agents known as calcium channel blockers. The contractile processes of smooth muscle cells are dependent upon calcium ions, which enter these cells during depolarisation as slow ionic transmembrane currents. Nimodipine inhibits calcium ion transfer into these cells and thus inhibits contractions of vascular smooth muscle.

Nimodipine is indicated for the improvement of neurological outcome by reducing the incidence and severity of ischemic deficits in patients with subarachnoid hemorrhage from ruptured intracranial berry aneurysms regardless of their post-ictus neurological condition. The precise mode of action is not clear. In patients with Hunt and Hess Grades I-III, nimodipine significantly reduces the risk of cerebral infarction and poor outcome in (subarachnoid hemorrhage) SAH. In patients with Hunt and Hess Grades IV and V, nimodipine improves recovery while decreasing severe disability and vegetative survival in SAH patients with poor neurological status.

Nimodipine is a yellow crystalline substance, practically insoluble in water. Currently, due to limited solubility, Nimodipine is available only as a soft-gel capsule, each capsule containing a 30 mg dose. As nimodipine is a substrate for cytochrome P450 3A4 isoenzyme and is thereby extensively and presystemically metabolized, resulting in a relative bioavailability of approximately 18%, a relatively high dose regime is required. Due to limited stability, one or two 30 mg large-soft gel capsules are administered up to six times per day, which constitutes a major inconvenience and leads to poor compliance.

A further difficulty is that, many patients who present with subarachnoid hemorrhage are variously incapacitated and require to be fed through naso-gastric tubing. As such patients are unable to swallow carers are required to syringe the contents of the soft-gel capsules out and to feed the drug solution through the feeding tube, a process that must be repeated up to six times per day.

To overcome the requirement to administer the nimodipine drug solution through naso-gastric tubing, many carers sought to circumvent this inconvenience by administering the drug intravenously. As nimodipine is a calcium channel blocker, in high, variable doses the potential to induce hypotension with potentially fatal consequences is very real. It was noted that a number of patients to whom nimodipine was administered intravenously died. In light of such deaths, the FDA has placed a black-box warning regarding administration errors.

In addition to the current subarachnoid hemorrhage indication, as an highly lipophilic calcium channel blocker that can pass the blood brain barrier and enter the cerebral vasculature, nimodipine, alone or in combination with other therapeutically active entities, may have a number of other activities in the brain, including cognitive enhancement, reducing neuropathic pain, alleviating stroke ailments, treating or preventing cluster headaches or migraines and preventing or treating neurodegenerative conditions, including Parkinson's Disease. Additionally, in combination with morphine nimodipine has been shown both to not only reduce the concentration of morphine required to reduce pain, but also extend the duration of pain reduction. None of the above potential indications is attractive if the drug requires to be administered up to six times a day and has a potentially fatal capacity to induce hypotension.

There is therefore a need for a formulation of nimodipine that will overcome at least some of these problems.

### STATEMENTS OF INVENTION

According to the invention there is provided a modified release solid dosage product comprising a plurality of minicapsules or minispheres containing nimodipine, wherein when exposed to a use environment more than 40% of the nimodipine is released within 12 hours and wherein the Tₘₐₓ is reached within 6 hours.

The product may comprise a first population of minispheres or minicapsules containing nimodipine and a second population of minispheres or minicapsules containing nimodipine.

The first population may comprise solid minicapsules or minispheres containing nimodipine for immediate release.

The second population may comprise minicapsules which are coated with a release agent. In one case the second population comprises minicapsules to release nimodipine over at least a 12 hour period. Alternatively or additionally the second population comprises minicapsules to release nimodipine over a 24 hour period.

In one embodiment the product comprises a first sub-population comprising minicapsules coated with a release agent to release nimodipine over a period of at least from 0 to 12 hours and a second sub-population to release nimodipine over a period of at least from 12 to 24 hours.

In a preferred embodiment the nimodipine is in a micronised form.

In one case less than 15% of the nimodipine is released within 1 hour. Less than 30% of the nimodipine may be released within 4 hours. Less than 35% of the nimodipine may be released within 6 hours.

In one embodiment the product is suitable for once daily administration. In this case the plasma concentration remains within 5 and 20 ng/ml for 75% of the time in a 24 hour period, preferably the plasma concentration remains within 7.5 and 15 ng/ml for 75% of the time in a 24 hour period.

The modified release dosage product may comprise from 90 mg to 450 mg of nimodipine, typically about 360 mg of nimodipine.

In one aspect of the invention provides a modified release solid dosage product comprising nimodipine wherein when exposed to a use environment more than 40% of the nimodipine is released within 6 hours and wherein the Tₘₐₓ is reached within 4 hours.

In one embodiment the product is suitable for twice daily administration. In this case the plasma concentration remains within 5 and 20 ng/ml for 75% of the time over a 12 hour period, preferably the plasma concentration remains within 7.5 and 15 ng/ml for 75% of the time over a 12 hour period.

The modified release dosage product may comprise from 45 mg to 210 mg of nimodipine, typically about 180 mg of nimodipine.

According to one aspect the invention provides modified release solid dosage product comprising nimodipine, wherein when exposed to a use environment more than 50% of the nimodipine is released within 12 hours and wherein the Tₘₐₓ is reached within 6 hours. In one embodiment substantially all of any remaining nimodipine is released between 12 and 24 hours. The product may comprise solid minicapsules containing nimodipine. The product may comprise one or more populations of minicapsules, at least one of which population comprising minicapsules which are coated with a release agent. The product is suitable for once daily administration. In one case the plasma concentration remains within 7.5 ng/ml and 15 ng/ml for 75% of the time in a 24 hour period. The modified release dosage product may comprise from 90 mg to 450 mg of nimodipine.

In one case the active pharmaceutical ingredient is an NO-donor conjugated Nimodipine.

In one embodiment the product is used to treat or prevent subarachnoid haemorrhage.

In another embodiment the product is used to treat or prevent stroke or transient ischemia.

In another embodiment the product is used to treat or prevent Alzheimer's Disease and other dementias, including but not limited to vascular dementia.

In another embodiment the product is used to treat or prevent neuropathic pain.

In a further embodiment the product is used to treat or prevent neurodegenerative disease. The neurodegenerative disease may be Parkinson's Disease, as Restless Leg Syndrome.

In another embodiment the product is used to treat amyotrophic lateral sclerosis.

In a further embodiment the product is used to treat Huntington's disease

In another embodiment the product is used to treat or prevent cluster headaches.

In a still further embodiment the product is used to treat or prevent migraine.

In another embodiment the product is used to treat or prevent bipolar disorder.

In another embodiment the product is used to treat or prevent schizophrenia.

In a further embodiment the product is used to treat or prevent preemclampsia.

In yet a further embodiment the product is used to treat or prevent epilepsy.

In another embodiment the product is used to treat or prevent Meniere's Disease. The product may be used to treat or prevent vertigo.

In one embodiment the product is a single-layer minicapsule containing Nimodipine or a NO-donor conjugate thereof and one or more other active pharmaceutical ingredient.

In one embodiment the gelling or encapsulating agent is gelatin, animal or non-animal derived.

In another embodiment the gelling or encapsulating agent is a non-gelatin entity, including, but not limited to, alginate, pectin, carrageenan or the like.

In another embodiment the product is a two-layer minicapsule. The core and shell may contain the same active pharmaceutical ingredient. Alternatively the core contains one or more active and the shell contains one or more different active. In one case the core formulation is controlled release and the shell is immediate release. In another case the core formulation is controlled release and the shell is controlled release.

In one embodiment the two-layer minicapsule is coated with a controlled release polymer or material.

In one aspect the product comprises at least one minicapsule population filled into hard gelatin capsules.

In another aspect the product comprises at least one minicapsule population filled into a sachet.

The product may comprise at least one minicapsule population contained within a wide gauge syringe or a unit that is compatible with tube delivery.

In another aspect the product comprises at least one minicapsule population in the form of a sprinkle.

At least one minicapsule population may be suspended in oil as a lubricant.

In one case the product comprises at least one minicapsule population formulated as a suppository for rectal or vaginal administration.

The product may comprise at least one minicapsule population formulated for buccal delivery.

The product may comprise at least one minicapsule population contained in a bioadhesive polymer strip.

In another case the product comprises at least one minicapsule population formulated for sublingual delivery.

At least one minicapsule population may be contained in a bioadhesive polymer strip.

In a further case the product comprises at least one minicapsule population contained in a sprinkle form.

The minicapsules may contain a disintegrant.

The minicapsules may contain a muco-adhesive or bio-adhesive.

The minicapsules may contain a permeability enhancer.

The minicapsules may contain a taste-masking agent.

In one embodiment the product comprises minispheres.

### BRIEF DESCRIPTION OF THE DRAWINGS

The invention will be more clearly understood from the following description of an embodiment thereof, given by way of example only, with reference to the accompanying drawings, in which:—

FIG. 1 illustrates the dissolution profile of an average of two batches from nimodipine solid minispheres over a 24 hour period. The profile represents release of 30 mg nimodipine from a blend of three distinct populations of minisphere: 5 mg uncoated, 6 mg coated with 15% weight gain Surelease® and 19 mg coated with 30% weight gain Surelease®. This product profile is suited to once-daily administration of nimodipine;

FIG. 2 illustrates the dissolution profile of an average of two batches from nimodipine solid minispheres over a 24 hour period. The profile represents release of 30 mg nimodipine from a blend of two distinct populations of minisphere: 9 mg uncoated and 21 mg coated with 20% weight gain Surelease®. This product profile is suited to twice daily administration of nimodipine;

FIG. 3 illustrates the dissolution profile of an average of two batches from nimodipine solid minispheres over a 24 hour period. The profile represents release of 30 mg nimodipine from a blend of two distinct populations of minisphere: 9 mg uncoated and 21 mg coated with 15% weight gain Surelease®. This product profile is suited to twice daily administration of nimodipine;

FIG. 4 illustrates the dissolution profile of an average of six batches from nimodipine solid minispheres over a 24 hour period. The profile represents release of 180 mg nimodipine from a blend of three distinct populations of minisphere: 14.9 mg uncoated, 35.6 mg coated with 7.5% weight gain Surelease® and 130.5 mg coated with 30% weight gain Surelease®. This product profile is suited to once-daily administration of nimodipine;

FIG. 5 illustrates the dissolution profile from an average of two batches of 30 mg 3-layer nimodipine uncoated minicapsules. The profile demonstrates that the core formulation is inherently sustained release;

FIG. 6 illustrates the dissolution profile from an average of two batches of 30 mg 3-layer nimodipine minicapsules over 24 hours. The 3-layer minicapsules were coated with a 6.5% weight gain blend of Eudragit® RS and Eudragit® RL to provide external controlled release as well as the inherent internal sustained release inherent to such 3-layer minicapsules, as demonstrated in FIG. 4;

FIG. 7 illustrates the dissolution profile from an average of two batches of 30 mg 3-layer nimodipine minicapsules over 24 hours. The 3-layer minicapsules were coated with a 13.5% weight gain blend of Eudragit® RS and Eudragit® RL to provide external controlled release as well as the inherent internal sustained release inherent to such 3-layer minicapsules, as demonstrated in FIG. 4;

FIG. 8 illustrates the pharmacokinetic plasma profile for the test product (180 mg Nimodipine as per FIG. 4) versus 6×30 mg Nimotop™ over a 24 hour period. The pharmacokinetic study represents the average of 20 healthy male volunteers and the plasma concentration is measured in ng/ml. This product profile is suited to once- or twice-daily administration; and

FIG. 9 illustrates schematically a population of individual solid, gelatine-based uncoated minispheres 1 encapsulating the micronized nimodipine. In the case illustrated there are two populations of variably weight-gain Surelease® polymer coated minispheres, 2 represents first lower weight gain Surelease® coated minispheres and 3 represents second higher weight gain Surelease® coated minispheres. The individual uncoated minispheres 1, lower weight gain Surelease® coated minispheres 2 and higher weight gain Surelease™ coated minispheres 3, are blended and filled into the final dosage form, in this instance, a two-cap, hard gelatine capsule 4.

### DETAILED DESCRIPTION

Oral drug modified release formats enable the provision of a means to control either where, when or how a drug is first released into the intestine and thereafter into the bloodstream to reach its desired target or locally along the gastrointestinal tract where it will act. Overall, compared with immediate release formats, modified release systems should ensure better disease management through steady state release or release to coincide with when a patient is most at risk and more convenience to the patient as the number of administrations per day is fewer and side effects are less pronounced. Based on the need for such dosage forms a number of modified release systems has been developed by those skilled in the art.

To date most modified release formats have been based on the development of various pellet, pill or particulate formats of varying shape and size, either coated with or incorporating the active drug, that are coated with any one of a number of polymers to modify the release profile. Such coatings are degraded by different environments, including pH change, time exposed to an aqueous solution or exposure to bacteria. Once the outer layer is compromised the drug inside, if soluble in an aqueous environment, will begin dissolving and start diffusing out into the intestinal lumen. The rate of dissolution and release is controlled mostly by the entry of gastric and intestinal fluids through the outer polymer shell and the inherent aqueous solubility of the drug in the core.

As the solubility of the drug is a major contributing factor to its dissolution, drugs with limited solubility are more difficult to formulate into existing modified release formats. As such drugs exhibiting low solubility have not been well catered for by existing modified release formats. Nimodipine is a poor water soluble, highly lipophilic drug, and the current administration format requires that it is first made soluble using oils and surfactants and then encapsulated into a large soft gel capsule format. The large soft gel capsule format is not suited to coating with modified release polymers or similar controlled release formulations. Thus, there is a need in the art for a controlled release system for nimodipine that will prevent plasma concentration peaks or troughs and ensure a steady therapeutic plasma concentration is maintained throughout a 24 hour period. The current invention details the development of controlled release minicapsule or minisphere nimodipine formulations that enable sustained release over a 24 hour period to permit once-daily or twice daily administration. Additionally, the current invention permits the development of novel controlled release combination products as potential therapeutics across a range of disease states.

The principle of seamless minicapsule formation is the utilisation of surface tension of one or more different solutions which when ejected through an orifice or nozzle with a certain diameter and subject to specific frequencies and gravitational flow, forms into a spherical form and falls into a cooling air flow or into a cooling or hardening solution and the outer shell solution where it is gelled or solidified. This briefly describes the formation of seamless minispheres.

According to prior art the core solution is mainly a hydrophobic solution or suspension. The outer shell solution can be any gel forming agent but is normally gelatin based but may also include polymers or other materials that enable controlled release. However a hydrophilic solution can also be encapsulated with the existence of an intermediate solution, which can avoid the direct contact of the hydrophilic core solution with the outer shell. With the nozzle having a single orifice, a minicapsule or a bead of shell/core mixed suspension of micronized drug can be processed. With the nozzle having two orifices (centre and outer), a hydrophobic solution can be encapsulated. With the nozzle having one or orifices seamless minicapsules for various applications can be processed. (Ref U.S. Pat. Nos. 5,882,680 and 6,312,942) Other encapsulation technologies such as those developed by Inotech, ITAS, including the Globex encapsulator may be used.

By using the above described manufacturing processing method as per U.S. Pat. No. 5,882,680 for multiparticulate seamless minicapsules, Nimodipine multiparticulate seamless minicapsules were produced. The completed Nimodipine seamless minicapsules preferably have an average diameter of 1.00-3.00 mm, more especially in the range 1.50-1.80 mm as described in our WO2006/035417A.

The resulting one-, two- or three-layer minicapsules or minispheres may be further processed to be coated with various controlled release polymers which modulates the release of active pharmaceutical actives from the underlying minicapsule or minisphere cores. In accordance with previous inventions the drug loaded minicapsules are coated with the rate-controlling polymers to achieve a target dissolution rate. The drug released from these minicapsules is diffusion controlled as the polymer swells and becomes permeable, it allows for the controlled release in the GIT. In order to achieve a suitable dissolution profile, the following parameters require consideration, efficient process/conditions, drug solubility/particle size, minicapsule surface area, minicapsule diameter and coating polymer suitability.

Additionally, certain semi-solid core formulations may result in controlled release alone or in conjunction with the shell, controlled release shell and/or controlled release shell coating.

### Controlled Release Polymers—Membrane-Controlled Dosage Forms

The modified-release formulations of the present invention can also be provided as membrane-controlled formulations. Membrane-controlled formulations of the present disclosure can be made by preparing a rapid release core, which can be liquid, semi-solid or solid, encapsulated by a gelatin shell, and coating the shell a functional coating. In the presence or absence of the membrane-controlled coating, the core, whether liquid, semi-solid or solid, can be formulated such that it itself controlled the release rate of the pharmaceutical compound from the minicapsules Details of membrane-controlled dosage forms are provided below.

In certain embodiments of the current invention, the pharmaceutical compound is provided in a multiple minicapsule membrane-controlled formulation. The active pharmaceutical can be formulated as a liquid, semi-solid or solid entity to enhance solubility, permeability or dissolution rate and utilized as the core of a two- or three-layer minicapsule that additionally comprises a shell with or without an additional buffer layer between to separate miscible core and shell constituents. The minicapsule diameter may range from 0.5 to about 5.0 mm. Additional pharmaceutical compound of the same active or one or more other actives can be sprayed from solution or suspension using a fluidized-bed coater or pan coating system.

To control the location of formulation release from the minicapsules, various delayed-release and/or extended-release polymeric materials, applied as a membrane coating to the minicapsules. The polymeric materials include both water-soluble and water-insoluble polymers. Possible water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof. Possible water-insoluble polymers include, but are not limited to, ethylcellulose, cellulose acetate, cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride), or polyurethane, and/or mixtures thereof.

EUDRAGIT® polymers (available from Evonik) are polymeric lacquer substances based on acrylates and/or methacrylates. A suitable polymer that is freely permeable to the active ingredient and water is EUDRAGIT® RL. A suitable polymer that is slightly permeable to the active ingredient and water is EUDRAGIT® RS. Other suitable polymers that are slightly permeable to the active ingredient and water, and exhibit a pH-dependent permeability include, but are not limited to, EUDRAGIT® L, EUDRAGIT® S, and EUDRAGIT® E.

EUDRAGIT® RL and RS are acrylic resins comprising copolymers of acrylic and methacrylic acid esters with a low content of quaternary ammonium groups. The ammonium groups are present as salts and give rise to the permeability of the lacquer films. EUDRAGIT® RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT® L is an anionic polymer synthesized from methacrylic acid and methacrylic acid methyl ester. It is insoluble in acids and pure water. It becomes soluble in neutral to weakly alkaline conditions. The permeability of EUDRAGIT® L is pH dependent. Above pH 5.0, the polymer becomes increasingly permeable.

In various embodiments comprising a membrane-controlled dosage form, the polymeric material comprises methacrylic acid co-polymers, ammonio methacrylate co-polymers, or mixtures thereof. Methacrylic acid co-polymers such as EUDRAGIT® S and EUDRAGIT® L (Evonik) are suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. EUDRAGIT® S and EUDRAGIT® L can be used as single components in the polymer coating or in combination in any ratio. By using a combination of the polymers, the polymeric material can exhibit solubility at a pH between the pHs at which EUDRAGIT® L and EUDRAGIT® S are separately soluble.

The membrane coating can comprise a polymeric material comprising a major proportion (i.e., greater than 50% of the total polymeric content) of at least one pharmaceutically acceptable water-soluble polymers, and optionally a minor proportion (i.e., less than 50% of the total polymeric content) of at least one pharmaceutically acceptable water insoluble polymers. Alternatively, the membrane coating can comprise a polymeric material comprising a major proportion (i.e., greater than 50% of the total polymeric content) of at least one pharmaceutically acceptable water insoluble polymers, and optionally a minor proportion (i.e., less than 50% of the total polymeric content) of at least one pharmaceutically acceptable water-soluble polymer.

The amino methacrylate co-polymers can be combined in any desired ratio, and the ratio can be modified to modify the rate of drug release. For example, a ratio of EUDRAGIT® RS:EUDRAGIT® RL of 90:10 can be used. Alternatively, the ratio of EUDRAGIT® RS:EUDRAGIT® RL can be about 100:0 to about 80:20, or about 100:0 to about 90:10, or any ratio in between. In such formulations, the less permeable polymer EUDRAGIT® RS would generally comprise the majority of the polymeric material with the more soluble RL, when it dissolves, permitting creating gaps through which solutes can enter the core and dissolved pharmaceutical actives escape in a controlled manner.

The amino methacrylate co-polymers can be combined with the methacrylic acid co-polymers within the polymeric material in order to achieve the desired delay in the release of the drug. Ratios of ammonio methacrylate co-polymer (e.g., EUDRAGIT® RS) to methacrylic acid co-polymer in the range of about 99:1 to about 20:80 can be used. The two types of polymers can also be combined into the same polymeric material, or provided as separate coats that are applied to the core.

In addition to the EUDRAGIT® polymers discussed above, other enteric, or pH-dependent, polymers can be used. Such polymers can include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethylcellulose acetate succinate, starch acetate phthalate, amylose acetate phthalate, polyvinyl acetate phthalate, and polyvinyl butyrate phthalate.

Surelease®, an aqueous ethylcellulose dispersion, is a unique combination of film-forming polymer; plasticizer and stabilizers. Designed for sustained release and taste masking applications, Surelease® is an easy-to-use, totally aqueous coating system using ethylcellulose as the release rate controlling polymer. The dispersion provides the flexibility to adjust drug release rates with reproducible profiles that are relatively insensitive to pH.

The principal means of drug release is by diffusion through the Surelease® dispersion membrane and is directly controlled by film thickness. Increasing or decreasing the quantity of Surelease® applied can easily modify the rate of release.

With Surelease® dispersion, reproducible drug release profiles are consistent right through from development to scale-up and production processes. More information can be found on the Colorcon Inc website at www. Colorcon.com. Additionally, a further range of controlled release polymers may be used.

Additionally, alternative controlled release enabling polymers or other entities may be used alone or in combination with polymers such as those mentioned above, including but not limited to Eudragit® and Surelease® polymers. Alternatively, any blend of controlled release materials or polymers may be employed.

The coating membrane can further comprise at least one soluble excipient to increase the permeability of the polymeric material. Suitably, the at least one soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose, and sucrose, sugar alcohols such as lactitol, maltitol, mannitol, sorbitol, and xylitol, xanthan gum, dextrins, and maltodextrins. In some embodiments, polyvinyl pyrrolidone, mannitol, and/or polyethylene glycol can be used as soluble excipients. The at least one soluble excipient can be used in an amount ranging from about 1% to about 20% by weight, based on the total dry weight of the polymer. The coating process can be carried out by any suitable means, for example, by using a perforated pan system such as the GLATT, ACCELACOTA, Diosna and/or HICOATER processing equipment.

The modifications in the rates of release, such as to create a delay or extension in release, can be achieved in any number of ways. Mechanisms can be dependent or independent of local pH in the intestine, and can also rely on local enzymatic activity to achieve the desired effect. Examples of modified-release formulations are known in the art and are described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566.

With membrane-modified extended-release dosage forms, a semi-permeable membrane can surround the formulation containing the active substance of interest. Semi-permeable membranes include those that are permeable to a greater or lesser extent to both water and solute. This membrane can include water-insoluble and/or water-soluble polymers, and can exhibit pH-dependent and/or pH-independent solubility characteristics. Polymers of these types are described in detail below. Generally, the characteristics of the polymeric membrane, which may be determined by, e.g., the composition of the membrane, will determine the nature of release from the dosage form.

A number of modified dosage forms suitable for use are described below. A more detailed discussion of such forms can also be found in, for example The Handbook of Pharmaceutical Controlled Release Technology, D. L. Wise (ed.), Marcel Decker, Inc., New York (2000); and also in Treatise on Controlled Drug Delivery: Fundamentals, Optimization, and Applications, A. Kydonieus (ed.), Marcel Decker, Inc., New York, (1992), the relevant contents of each of which are hereby incorporated by reference for this purpose. Examples of modified-release formulations include but are not limited to, membrane-modified, matrix, osmotic, and ion-exchange systems. All of these can be in the form of single-unit or multi-unit dosage forms, as alluded to above.

The pH-dependent systems exploit the generally accepted view that pH of the human GIT increases progressively from the stomach (pH 1-2 which increases to 4 during digestion), small intestine (pH 6-7) at the site of digestion and it increases to 7-8 in the distal ileum. The coating of pH-sensitive polymers to the tablets, capsules or pellets provide delayed release and protect the active drug from gastric fluid. The polymers used for colon targeting, however, should be able to withstand the lower pH values of the stomach and of the proximal part of the small intestine and also be able to disintegrate at the neutral of slightly alkaline pH of the terminal ileum and preferably at the ileocecal junction.

While the minicapsule process above exhibits a number of benefits for a range of active pharmaceutical compounds potential limitations include compatibilities of core formulations with the shell material and/or the buffer layer, where required. Another potential limitation is low active pharmaceutical compound payloads leading to large, patient-unfriendly pill sizes. Still another potential limitation is that controlled release is a function of the shell or shell coating and may thus be limiting. Yet another limitation relates to possible incompatibilities between the shell and the core or the buffer layer which results in incomplete encapsulation or irregular shaped minicapsules. Still another advantage relates to the possibility to develop novel, otherwise incompatible, controlled release combination products for the potential treatment of an array of disease conditions.

### Calcium Regulation in Health and Disease

Calcium has a pervasive role in regulating brain function, for example plasticity, glucose metabolism, neurotransmitter synthesis and release, axonal transport and neuronal dendritic claw formation. Calcium ions are ubiquitous messengers linking membrane excitation to subsequent intracellular molecular responses. Changes in calcium homoeostasis are an aspect of aging that may have implications for higher cerebral functions.

Nimodipine is an isopropyl calcium channel blocker with lipophilic properties that permit is to readily cross the blood-brain barrier. Its primary action is to bind to L-type receptors and reduce the number of open channels conveying calcium ions through the cell membrane, thereby restricting influx of calcium ions into cells. It has anti-vasoconstrictive and vasodilatory action or arterioles.

The current invention seeks to capitalize on the benefits proffered through the minicapsule process described above through the development controlled release nimodipine formulations based on the minicapsule or minisphere process, either alone or in combination with other pharmaceutically active entities.

### Subarachnoid Hemorrhage

Nimodipine, when administered 60 milligrams every 4 hours should be initiated within 96 hours and continued for 21 days, is indicated to reduce the severity of ischemic neurological deficits in patients with subarachnoid hemorrhage including all Hunt and Hess grades [I through V] (Thomson Report, 2005).

The current invention will permit the development of once-daily or twice daily nimodipine for the treatment of subarachnoid hemorrhage. In addition to the added convenience of once-daily rather than six times daily treatment, the minicapsule format will be suited to easy administration through naso-gastric tubing, either with or without need for a funnel-like tube attachment.

### Stroke/Transient Ischemia

The present invention permits the development of once-daily or twice-daily, sustained release nimodipine for the treatment or prevention of stroke.

### Alzheimer's Disease/Dementia

In experimental situations inhibition of calcium entry can protect neurons against a range of damaging influences, and this suggests that nimodipine may have neuroprotective potential, particularly against ischemia and hypoxia. The density of nimodipine binding sites is especially high in specific regions of the hippocampus, caudate nucleus, and cerebral cortex, and may have a role in learning and memory processes. Binding sites are found on neurons and cerebrovascular cells, and its actions may influence both neuronal conduction and cerebral blood flow (Tedeschi, Cur Therap Res 1991; 50:553-63).

Unlike other calcium channel blockers, nimodipine produces its anti-vasoconstrictive and anti-ischemic effects primarily in the brain and at low doses, most evidently on smaller arterioles that determine regional blood flow. It can also modulate other calcium dependent processes such as acetylcholine release, potentially of benefit in improving functions in Alzheimer's Disease (Baumel et al, Diagnosis and treatment of senile dementia. Berlin Heidelberg: Springer-Verlag, 1989:366-73).

The present invention enables the development of once-daily or twice-daily, controlled release nimodipine for the treatment or prevention of Alzheimer's Disease or other forms of dementia.

### Neurodegenerative Diseases

Single neurons form thousands of specialized connections with other neurons called synapses. The number, strength, and specificity of these synaptic connections ultimately determine and regulate brain function. As such, how synaptic connectivity is established during development and how it is modified through life is important in regulating the maintenance of healthy minds or development of neurological or neurodegenerative diseases. The present invention enables the development of novel once-daily or twice-daily, controlled release nimodipine alone to effect channel blocking and calcium channel modulation activity for the treatment of Parkinson's Disease or other neurodegenerative diseases, such as but not limited to Restless Leg Syndrome.

### Depression/Bipolar

Adjunctive nimodipine may add to amelioration of depression in some patients with cerebrovascular disease. Also, a patient with bipolar disorder experienced satisfactory results with nimodipine, which was used during her pregnancy (Thomson Report, 2005).

The present invention enables the development of once-daily or twice daily controlled release nimodipine for the treatment of depression or bipolar disorders.

### Cluster Headache/Migraine

Nimodipine, in total daily doses of 60 to 120 milligrams, has been effective in the treatment of cluster headache (Thomson Report, 2005).

The present invention enables the development of once-daily or twice-daily controlled release nimodipine for the symptomatic treatment or prophylaxis of cluster headaches or migraines.

### Amyotrophic Lateral Sclerosis

Amyotrophic Lateral Sclerosis (ALS) is a progressive, fatal neurodegenerative disease caused by the degeneration of motor neurons. While the cause remains unknown, the role of glutamate and reactive oxygen species cannot be ruled out. The only drug approved for ALS is riluzole, which is believed to reduce damage to motor neurons by decreasing the release of glutamate. Further studies in mice showed a dramatic benefit when riluzole is combined with nimodipine.

The present invention permits the development of once-daily or twice-daily, controlled release nimodipine for the treatment of prevention of ALS.

### Huntington's Disease

Huntington's disease is a rare inherited neurological disorder caused by a trinucleotide repeat expansion in the Huntington gene which results in neuronal cell death in select areas of the brain and is a terminal illness. It is characterised by movement disorder, dementia and psychiatric disorders. Neuronal cell degeneration occurs primarily in the frontal lobes, the basal ganglia, and caudate nucleus. Potential treatments include nimodipine.

The present invention permits the development of once-daily or twice-daily, controlled release nimodipine alone for the treatment or prevention of Huntington's disease.

### Preemelampsia

Nimodipine has been used for the treatment of very high blood pressure during pregnancy (Duley and Henderson-Smart, Cochrane Database Syst Rev. 2002; CD001449).

The present invention enables the development of once-daily or twice-daily, controlled release nimodipine for the treatment or prevention of preemclampsia.

### Meniere's Disease

Nimodipine was investigated in patients with Meniere's disease for whom first-line medical management had failed. In two-thirds of patients, successful control of vertigo and hearing improvement or stabilisation was observed (Lassen et al., American Journal of Otology, 1996; 17(4):577-580).

The present invention enables the development of once-daily or twice-daily, controlled release nimodipine for the treatment of Meniere's Disease.

### Administration Formats

The multiple minicapsule or minisphere format enables combinations of one active with different controlled release coatings or alternatively different actives with single or multiple controlled release coatings to be filled into hard gelatine capsules of various sizes. The hard gelatine capsule may also contain liquid formulations or powder formulations. Furthermore, the minicapsules or minispheres may be compressed into pellet or pill format comprised or inactive excipients or other active pharmaceutical ingredients.

An advantage of the current minicapsule and minisphere forms is that they are format flexible leading to ease of administration. A common problem in many of the conditions with potential to be treated by nimodipine or combination products containing nimodipine is that patient's experience swallowing difficulties. This may arise due to a patient being incapacitated following a stroke or trauma and fed through a naso-gastric tube or in certain neurodegenerative diseases such as Parkinson's Disease where the patient may experience difficulty in swallowing.

In one easy to administer format, the present invention permits that the minicapsules or minispheres may be filled into sachets, the contents of which may be sprinkled onto soft food or, indeed, drinks and administered to patients by spoon feeding, drinking or through a straw. This form of administration is suited to paediatrics or geriatrics that dislike or have difficulty swallowing. Furthermore, the sachet contents may be poured into an attachment to naso-gastric tubing for administration to incapacitated patients. Another format is to pre-fill the contents into a syringe that may be connected to naso-gastic tubing.

Still another administration format is in suppository format that is suited to vaginal or rectal administration. This format has a number of advantages, including administration to patients in acute need for a rapid onset of action and may be incapable of swallowing.

Additionally, the minicapsules or minispheres may be incorporated into a format for buccal or sub-lingual administration. Such formats may include bioadhesive degradable films, including hydrogels or formats that may disintegrate rapidly in the mouth or under the tongue. Again, this format is suited to the need for a quick onset of action or for patients unable to swallow.

### Controlled Release Nimodipine

### Uncoated Minicapsules

Appropriate quantities of micronised nimodipine, gelatine and sorbitol are added to water and heated to 80° C., continually stirring until a homogeneous solution is achieved. The solution is then processed into solid minispheres at an appropriate flow rate and vibrational frequency using the manufacturing processing method described in U.S. Pat. No. 5,882,680 The resulting minispheres are cooled in oil. The cooled minispheres are harvested and centrifuged to remove residual oil and dried overnight in an oven. Nimodipine multiparticulate seamless minicapsules were produced. The completed Nimodipine seamless minicapsules had an average diameter in the range 1.50-1.80 mm.

| | |
|---|---|
| Ingredients | % w/w |

Core Composition

| | |
|---|---|
| Nimodipine (Micronised) | 37.5 |
| Gelatin | 56.3 |
| Sorbitol | 6.3 |

### Coated Minicapsules

Some of the uncoated minicapsules are coated with Surelease® using standard bottom spray fluidized bed coating, as enabled using a Diosna Minilab, to provide a 12-hour or a 24-hour release profile.

In one case the coating is a low weight gain Surelease® such as 7.5% wt gain Surelease®, Typically: curing 40° C.×24 hr. The dissolution profile is obtained by placing the resulting minicapsules in 0.3% SDS in Water, 100 rpm, HPLC—over 24 hr.

In another case the coating is a higher weight gain Surelease®, such as 30% wt gain Surelease®, Typically: curing 40° C.×24 hr. The dissolution profile is obtained by placing the resulting minicapsules in 0.3% SDS in Water, 100 rpm, HPLC—over 24 hr.

### Final Dosage Form

The uncoated minispheres and one or more populations of coated minispheres are blended and filled into the final dosage form.

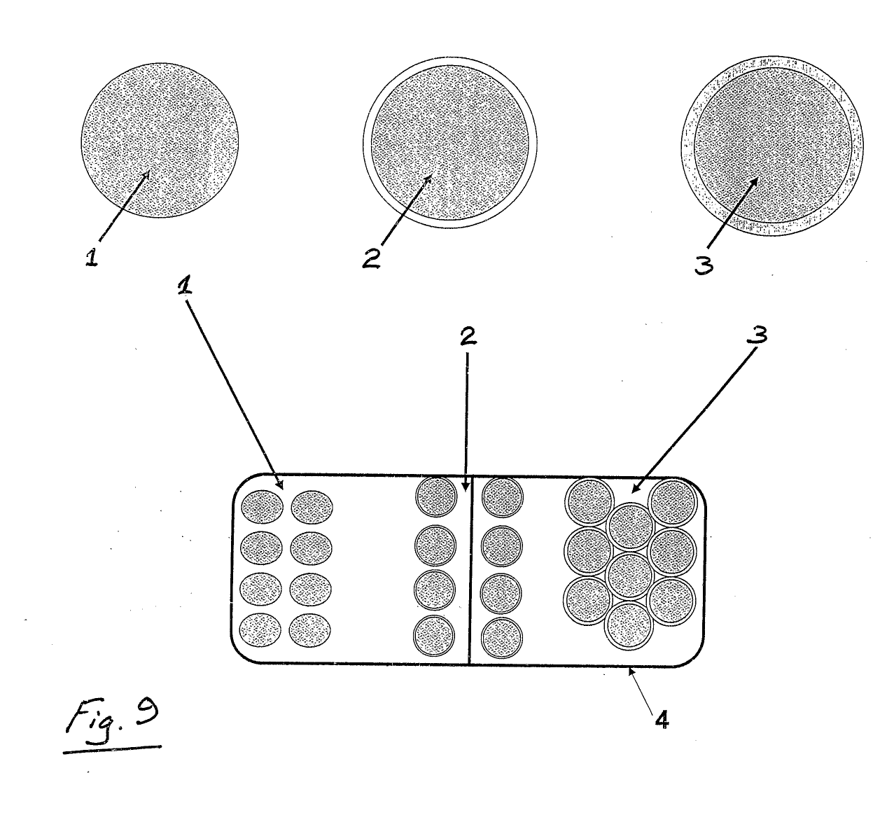

In more detail, FIG. 9 illustrates schematically a population of individual solid, gelatine-based uncoated minispheres 1 encapsulating the micronized nimodipine. In the case illustrated there are two populations of variably weight-gain Surelease® polymer coated minispheres, 2 represents first lower weight gain Surelease® coated minispheres and 3 represents second higher weight gain Surelease® coated minispheres. The individual uncoated minispheres 1, lower weight gain Surelease® coated minispheres 2 and higher weight gain Surelease® coated minispheres 3, are blended and filled into the final dosage form, in this instance, a two-cap, hard gelatine capsule 4.

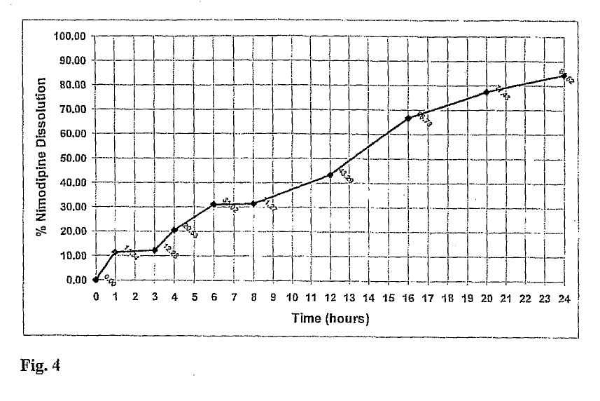

24-hour nimodipine dissolution data is presented in Table 4 and the dissolution profile is graphically illustrated in FIG. 4.

### Example 1

### Nimodipine QD1 Formulation

Using the manufacturing process described above a nimodipine QD1 formulation (30 mg) was prepared from a blend of 5 mg Uncoated, 6 mg 15% wt gain, 19 mg 30% wt gain Surelease, Curing 40° C.×24 hr. The dissolution profile is obtained by placing the resulting minicapsules in 0.3% SDS in Water, 100 rpm, HPLC—over 24 hr.

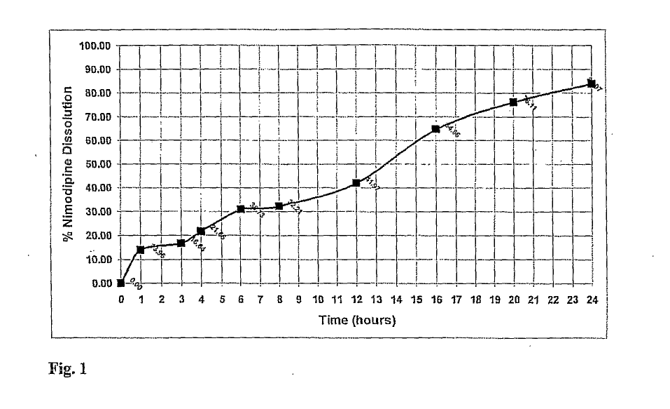

TABLE 1
Release of Nimodipine QD1 Formulation (30 mg) - Blend of 5 mg
Uncoated, 6 mg 15% wt gain, 19 mg 30% wt gain Surelease, Curing
40° C. × 24 hr. The dissolution profile is obtained by placing
the resulting minicapsules in 0.3% SDS in Water, 100 rpm,
HPLC - over 24 hr. The release profile is illustrated in FIG. 1.

| | | |
|---|---|---|
| Time | Dissolution: % Release | Average |

| | | | |
|---|---|---|---|
| 0 | 0.00 | 0.00 | 0.00 |
| 1 | 14.88 | 13.04 | 13.96 |
| 3 | 15.80 | 17.47 | 16.64 |
| 4 | 20.52 | 22.79 | 21.66 |
| 6 | 29.75 | 31.70 | 30.73 |
| 8 | 32.44 | 31.97 | 32.21 |
| 12 | 43.34 | 40.60 | 41.97 |
| 16 | 64.13 | 65.58 | 64.86 |
| 20 | 73.86 | 78.36 | 76.11 |
| 24 | 80.56 | 87.37 | 83.97 |

### Example 2

### Nimodipine BID 1 Formulation

Using the manufacturing process described above a nimodipine BID 1 formulation (30 mg) was prepared from a blend of 9 mg uncoated, 21 mg 15% wt gain Surelease, Curing 40° C.×24 hr. The dissolution profile is obtained by placing the resulting minicapsules in 0.3% SDS in Water, 100 rpm, HPLC—over 24 hr.

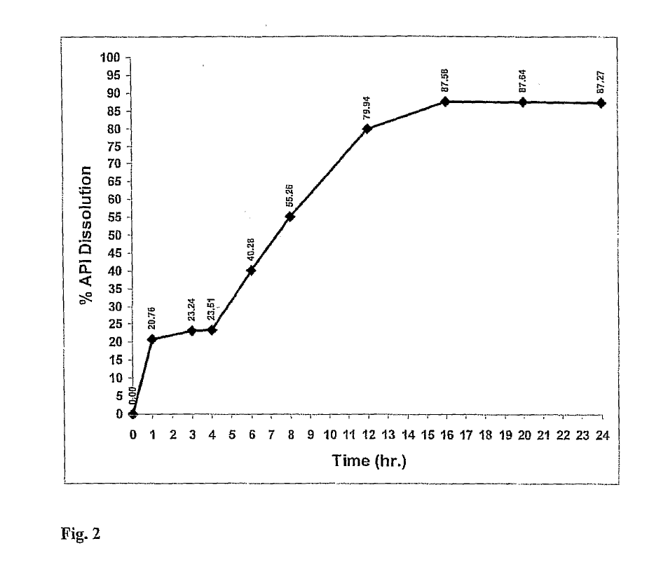

TABLE 2
Release of Nimodipine BID 1 Formulation (30 mg) - Blend of 9 mg
Uncoated, 21 mg 15% wt gain Surelease, Curing 40° C. × 24 hr.
The dissolution profile is obtained by placing the resulting
minicapsules in 0.3% SDS in Water, 100 rpm, HPLC - over 24 hr.
The release profile is illustrated in FIG. 2.

| | | |
|---|---|---|
| Time | Dissolution % Release | Average |

| | | | |
|---|---|---|---|
| 0 | 0.00 | 0.00 | 0.00 |
| 1 | 21.76 | 19.75 | 20.76 |
| 3 | 22.64 | 23.84 | 23.24 |
| 4 | 23.11 | 23.90 | 23.51 |
| 6 | 42.63 | 37.93 | 40.28 |
| 8 | 55.93 | 54.58 | 55.26 |
| 12 | 80.17 | 79.71 | 79.94 |
| 16 | 85.69 | 89.47 | 87.58 |
| 20 | 86.16 | 89.12 | 87.64 |
| 24 | 85.24 | 89.30 | 87.27 |

### Example 3

### Nimodipine BID 1 Formulation

Using the manufacturing process described above a nimodipine BID 1 formulation (30 mg) was prepared from a blend of 9 mg Uncoated, 21 mg 20% wt gain Surelease, Curing 40° C.×24 hr. The dissolution profile is obtained by placing the resulting minicapsules in 0.3% SDS in Water, 100 rpm, HPLC—over 24 hr.

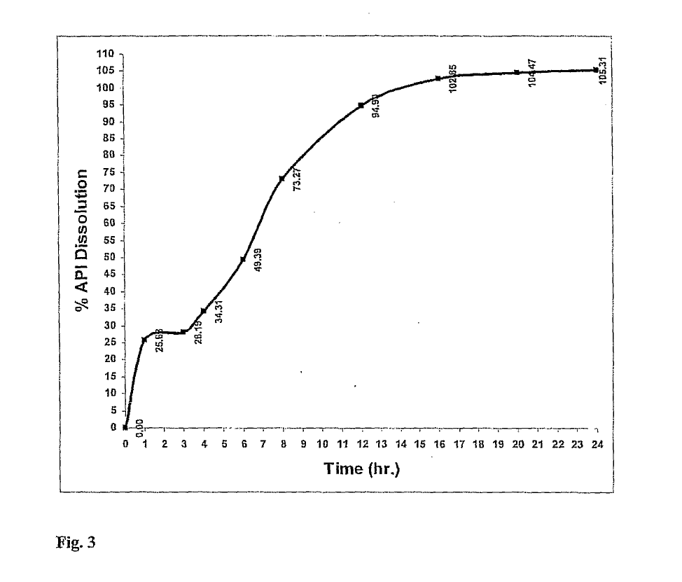

TABLE 3
Release of Nimodipine BID 1 Formulation (30 mg) - Blend of 9 mg
Uncoated, 21 mg 20% wt gain Surelease, Curing 40° C. × 24 hr.
The dissolution profile is obtained by placing the resulting
minicapsules in 0.3% SDS in Water, 100 rpm, HPLC - over 24 hr.
The release profile is illustrated in FIG. 3.

| | | |
|---|---|---|
| Time | Dissolution: % Release | Average |

| | | | |
|---|---|---|---|
| 0 | 0.00 | 0.00 | 0.00 |
| 1 | 27.28 | 27.08 | 22.67 |
| 3 | 27.86 | 28.78 | 27.94 |
| 4 | 33.95 | 36.65 | 32.33 |
| 6 | 49.62 | 55.94 | 42.60 |
| 8 | 72.78 | 80.74 | 66.29 |
| 12 | 96.66 | 96.66 | 91.39 |
| 16 | 101.87 | 104.32 | 102.37 |
| 20 | 104.38 | 104.38 | 104.65 |
| 24 | 106.13 | 105.36 | 104.44 |

### Example 4

### Nimodipine QD1 Formulation

Using the manufacturing process described above a nimodipine QD1 formulation (30 mg) was prepared from a blend of 14.9 mg uncoated, 35.6 mg 7.5% wt gain Surelease®, 130.5 mg 30% wt gain Surelease®, Curing 40° C.×24 hr. The dissolution profile is obtained by placing the resulting minicapsules in 0.3% SDS in Water, 100 rpm, HPLC—over 24 hr. The individual uncoated minispheres 1, lower weight gain Surelease® coated minispheres 2 and higher weight gain Surelease™ coated minispheres 3, are blended and filled into the final dosage form, in this instance, a two-cap, hard gelatine capsule 4 as illustrated in FIG. 9.

TABLE 4
Release of Nimodipine QD Formulation (180 mg) - Blend of
14.9 mg Uncoated, 35.6 mg 7.5% wt gain Surelease ®,
130.5 mg 30% wt gain Surelease ®, Curing 40° C. × 24 hr.
The dissolution profile is obtained by placing the resulting
minicapsules in 0.3% SDS in Water, 100 rpm, HPLC - over 24 hr.
The release profile is illustrated in FIG. 4.

| Time | Dissolution: % Release | | | Average |
|---|---|---|---|---|
| 0 | 0.00 | 0.00 | 0.00 | 0.00 |
| 1 | 12.03 | 11.03 | 10.96 | 11.34 |
| 3 | 12.04 | 11.96 | 12.85 | 12.28 |
| 4 | 19.46 | 20.24 | 21.90 | 20.53 |
| 6 | 30.67 | 30.43 | 31.95 | 31.02 |
| 8 | 30.81 | 30.79 | 32.21 | 31.27 |
| 12 | 41.21 | 42.02 | 46.63 | 43.29 |
| 16 | 65.53 | 65.81 | 68.86 | 66.73 |
| 20 | 76.04 | 78.34 | 77.91 | 77.43 |
| 24 | 85.10 | 84.03 | 84.72 | 84.62 |

### Example 5

### Controlled Release Nimodipine Human Study

A test product—a single capsule containing 180 mg Nimodipine as per example 4 was administered to healthy male volunteers. The results were compared against administration of 6×30 mg known formulations of nimodipine —Nimotop™ over a 24 hour period. The pharmacokinetic study represented the average of 20 healthy male volunteers and the plasma concentration was measured in ng/ml.

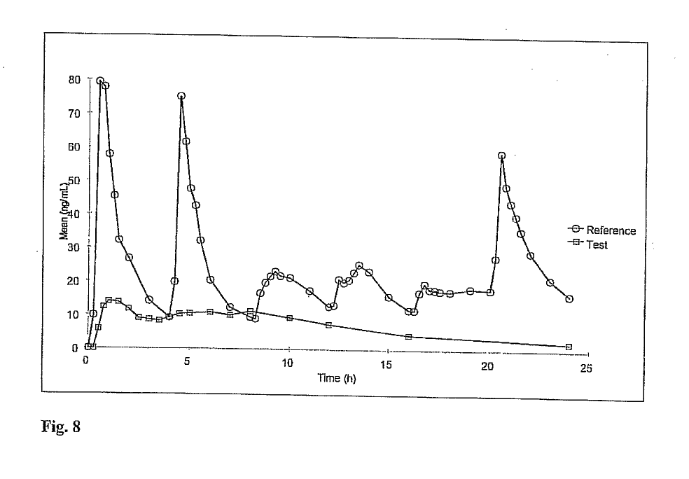

FIG. 8 illustrates the pharmacokinetic plasma profile for the test product (180 mg Nimodipine as per example 4 versus 6×30 mg Nimotop™ over a 24 hour period. The pharmacokinetic study represents the average of 20 healthy male volunteers and the plasma concentration is measured in ng/ml.

This product profile is suited to once- or twice-daily administration.

### Example 6

### Controlled Release Three-Layer Nimodipine Minicapsules

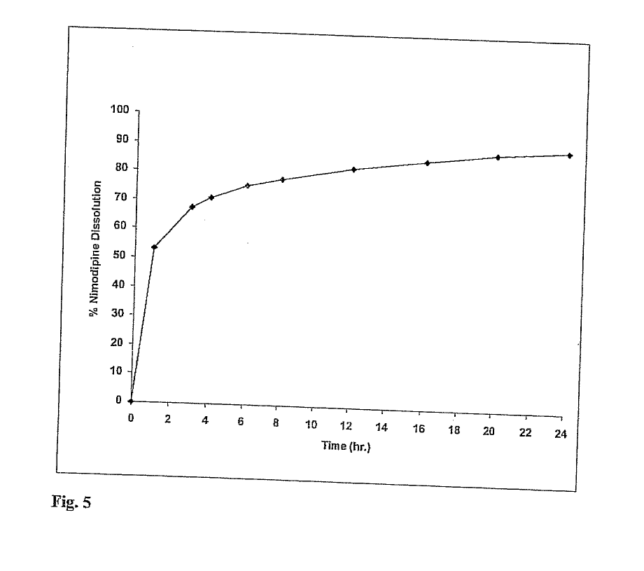

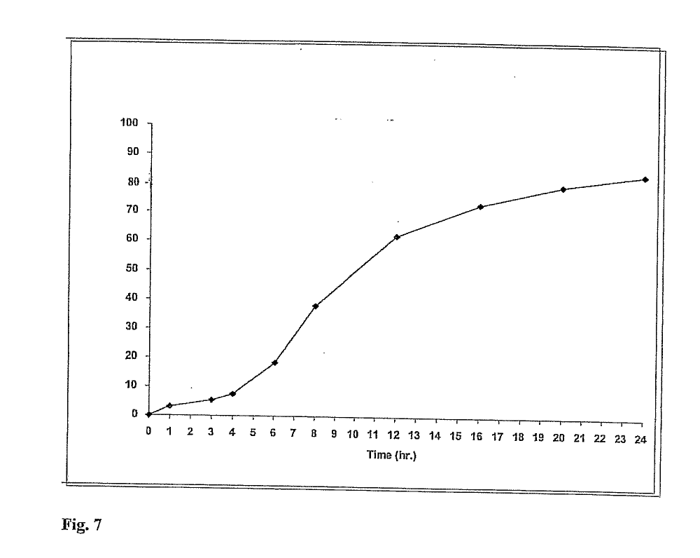

An appropriate quantity of nimodipine is added to PEG 400, heated and stirred until the nimodipine is fully dissolved. The solution is then processed to flow through the central nozzle of a tri-centric nozzle with heated gelucire passing through the middle nozzle and a molten gelatine/sorbitol solution passed through the outer nozzle. The three solutions are passed through the tri-centric nozzle with each flowing at appropriate flow rates and vibrational frequency. The resulting three-layer minicapsules are cooled in oil. The cooled minispheres are harvested and centrifuged to remove residual oil and dried overnight in an oven. The resulting minicapsules are further coating with either a 6.5% or 13.5% weight gain 50:50 Eudragit® RS/Eudragit® RL to provide a 24-hour release profile. The uncoated 3-layer nimodipine 24 hour dissolution data is presented in Table 5 and the related dissolution profile is graphically illustrated in FIG. 5. The Nimodipine 3 Layer Formulation 6.5% weight gain 50:50 Eudragit RS/RL 24 hour dissolution data is presented in Table 6 and the related dissolution profile is graphically illustrated in FIG. 7. The Nimodipine 3 Layer Formulation 13.5% weight gain 50:50 Eudragit RS/RL 24 hour dissolution data is presented in Table 7 and the related dissolution profile is graphically illustrated in FIG. 7.

| | |
|---|---|
| Ingredients | % w/w |

Core Composition

| | |
|---|---|
| Nimodipine | 8.5 |
| PEG 400 | 91.5 |

Mid-Layer Composition

| | |
|---|---|
| Gelucire 33/1 | 100 |

Shell Composition

| | |
|---|---|
| Sorbitol | 10 |
| Gelatin | 90 |

TABLE 5
Nimodipine 3 Layer Formulation Uncoated (30 mg) - 0.3% SDS in
Water, 100 rpm, HPLC - over 24 hr. The release profile is
illustrated in FIG. 5.

| Dissolution (%) | | | |
|---|---|---|---|
| Time (hours) | N = 1 | N = 2 | Average |
| 0 | 0.00 | 0.00 | 0.00 |
| 1 | 53.29 | 52.88 | 53.09 |
| 3 | 67.96 | 66.94 | 67.45 |
| 4 | 71.09 | 70.42 | 70.76 |
| 6 | 75.56 | 74.78 | 75.17 |
| 8 | 78.13 | 77.84 | 77.99 |
| 12 | 82.54 | 82.18 | 82.36 |
| 16 | 85.32 | 85.39 | 85.36 |
| 20 | 87.96 | 88.35 | 88.16 |
| 24 | 89.59 | 90.43 | 90.01 |

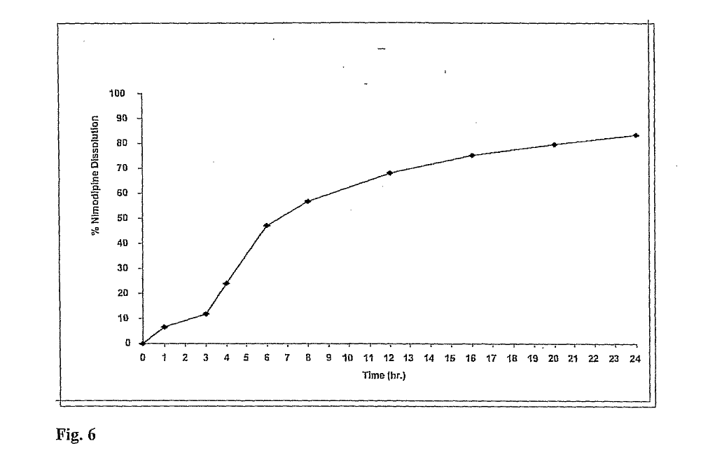

TABLE 6
Nimodipine 3 Layer Formulation 6.5% wt gain 50:50 Eudragit
RS/RL (30 mg) - Curing 40° C. × 24 hr, 0.3% SDS in Water,
100 rpm, HPLC - over 24 hr. The release profile is illustrated in FIG. 6.

| Dissolution (%) | | | |
|---|---|---|---|
| Time (hours) | N = 1 | N = 2 | Average |
| 0 | 0.00 | 0.00 | 0.00 |
| 1 | 6.84 | 6.04 | 6.44 |
| 3 | 12.71 | 10.85 | 11.78 |
| 4 | 24.98 | 23.04 | 24.01 |
| 6 | 47.73 | 46.89 | 47.31 |
| 8 | 55.68 | 58.12 | 56.90 |
| 12 | 66.48 | 69.60 | 68.04 |
| 16 | 73.04 | 77.28 | 75.16 |
| 20 | 77.92 | 81.75 | 79.84 |
| 24 | 81.06 | 85.65 | 83.36 |

TABLE 7
Nimodipine 3 Layer Formulation 13.5% wt gain 50:50 Eudragit RS/RL
(30 mg) - Curing 40° C. × 24 hr, 0.3% SDS in Water,
100 rpm, HPLC - over 24 hr. The release profile is illustrated in FIG. 7.

| Dissolution (%) | | | |
|---|---|---|---|
| Time (hours) | N = 1 | N = 2 | Average |
| 0 | 0.00 | 0.00 | 0.00 |
| 1 | 3.07 | 3.41 | 3.24 |
| 3 | 5.06 | 5.74 | 5.40 |
| 4 | 6.61 | 8.60 | 7.61 |
| 6 | 14.75 | 22.26 | 18.51 |
| 8 | 35.72 | 40.37 | 38.05 |
| 12 | 61.11 | 63.28 | 62.20 |
| 16 | 72.63 | 73.70 | 73.17 |
| 20 | 78.72 | 80.65 | 79.69 |
| 24 | 82.93 | 84.46 | 83.70 |

### Example 6

### Controlled Release Nimodipine (with Vitamin E for Enhanced Bioavailability)

Appropriate quantities of micronised nimodipine, gelatine, sorbitol and vitamin E are added to water and heated to 80° C., continually stirring until in a homogeneous solution. The solution is then processed into solid minispheres at an appropriate flow rate and vibrational frequency. The resulting minispheres are cooled in oil. The cooled minispheres are harvested and centrifuged to remove residual oil and dried overnight in an oven. The resulting minicapsules are further coating using Surelease® to provide a 12-hour or a 24-hour release profile.

| | |
|---|---|
| Ingredients | % w/w |

Core Composition

| | |
|---|---|
| Nimodipine | 37.5 |
| Vitamin E | 4.7 |
| Gelatin | 51.6 |
| Sorbitol | 6.3 |

The invention is not limited to the embodiments hereinbefore described which may be varied in detail.

1. A modified release solid dosage product comprising a plurality of minicapsules or minispheres containing nimodipine, wherein when exposed to a use environment more than 40% of the nimodipine is released within 12 hours and wherein the Tₘₐₓ is reached within 6 hours.

2-54. (canceled)
